(19) United States
(12) Patent Application Publication
(10) Pub. No.: US 2025/0352445 A1
(43) Pub. Date: Nov. 20, 2025

(54) COMPOSITIONS FOR SEBUM CONTROL

(71) Applicant: Dyson Operations Pte. Ltd., Singapore (SG)

(72) Inventors: Maurice Adrianus Monique VAN STEENSEL, Singapore (SG); Kai Xuan Keith TAN, Singapore (SG); Jun Xi Selwyn LOW, Singapore (SG); Liang DAI, Singapore (SG); Hwei-In Shawn LIM, Singapore (SG)

(21) Appl. No.: 18/872,338

(22) PCT Filed: Jun. 7, 2023
(86) PCT No.: PCT/IB2023/055872

§ 371 Date: Dec. 6, 2024

(30) Foreign Application Priority Data
Jun. 7, 2022 (GB) 2208320.8

(51) Int. Cl. A61K 8/36; A61K 9/00; A61K 31/19; A61K 31/22; A61K 36/18; A61Q 19/00

(52) U.S. Cl. CPC A61K 8/361; A61K 9/0014; A61K 31/19; A61K 31/22; A61K 36/18; A61Q 19/008

(57) ABSTRACT

The invention provides a composition for use in decreasing lipid production in sebocytes or in the skin of an individual, where the composition includes a triterpenoid compound as shown in formula (I), as described herein, or a pharmaceutically or cosmetically acceptable salt, prodrug, solvate, tautomer or stereoisomer thereof; and a plant extract selected from extracts of plants belonging to the genus Garcinia, Rubus, Fragaria, Ilex and Hedera.

### FIELD OF THE INVENTION

The invention relates to compositions comprising a plant extract in combination with triterpenoid compounds, and the use of the composition in a method for decreasing lipid production in sebocytes. Also provided is the use of the composition in a cosmetic method for decreasing lipid production in the skin of an individual, and the use of the composition in a method of treating a skin disease or disorder related to increased lipid production in sebocytes.

### BACKGROUND

Sebum is an oily substance secreted by sebaceous glands in humans. Sebum is produced by sebocytes, highly-specialised epithelial cells commonly found in the skin in association with hair follicles, although there are also sebaceous glands not associated with hair follicles.

Adult human sebum typically comprises triglycerides (˜41%), wax monoesters (˜25%), free fatty acids (˜16%), and squalene (˜12%) (Cheng et al., 2004). Other components, such as keratin and cellular materials, may also be present.

Sebum forms an integral component of the epidermal barrier and the skin immune system. Sebum is a natural moisturizer for the epidermis, helping to maintain its integrity. Sebum is important in maintaining the pH of the skin surface, which may play a role in protecting the skin from exogenous (disease-causing) microbes and encouraging the growth of endogenous (resident) microflora. Sebaceous secretions in conjunction with apocrine (sweat) glands are also thought to play an important thermoregulatory role.

Sebocyte formation is controlled by multiple molecular pathways (e.g. Blimp1, Wnt, C-myc, Hedgehog) and sebum synthesis is strongly regulated by hormones, in particular by androgens such as testosterone.

Sebum is produced in a holocrine process, in which sebocytes rupture and disintegrate as they release the sebum along with cell remnants. During the terminal differentiation of sebocytes, metabolic activity is concentrated on the biosynthesis of lipids (lipogenesis), and in particular on the neosynthesis of fatty acids and squalene.

The level of sebum production varies from person to person and is influenced by sex, age, physical activity, stress, certain medications, and disease. Oily skin is commonly observed in adolescence due to hormonal changes occurring throughout puberty.

Excessive sebum production is associated with cosmetic problems, such as oily or shiny skin and poor retention of make-up, as well as medical problems. Excessive sebum production is seen in acne vulgaris, one of the most common skin diseases. Hyperseborrhea is a scalp problem cause by excessive production of sebum. Immediate symptoms of hyperseborrhea include scalp itchiness and pain, though later symptom is hair loss. Individuals with hyperseborrhoeic skin typically exhibit sebum levels of greater than 200 μg cm⁻² measured on the forehead (as discussed, for example, in WO 2020/263188). Deregulated sebocyte differentiation also characterizes some rare benign and malignant tumors.

Cosmetic treatments for excess lipid production generally do not address the underlying causes. Rather, cosmetic treatments typically provide relief from the direct symptoms, such as oiliness, enlarged pores, acne prone skin, and irregular skin texture. For example, a common approach to treating oily or shiny skin is the use of powders that provide an immediate masking effect by absorbing the excess sebum on the skin's surface. Alternatively, astringents and cleaning agents may be used.

The known methods for reducing lipids on the skin surface are limited, producing little sustainable visible results over extended periods of time. Prolonged use of astringents and cleaning agents may exacerbate the condition.

Accordingly, there is a need to develop compositions and methods for reducing lipid production in sebocytes and in the skin of an individual.

### SUMMARY OF THE INVENTION

At its most general, the invention relates to a composition comprising a plant extract in combination with a triterpenoid compound. The inventors have found that such a composition provides superior lipid reducing effects in sebocytes.

The triterpenoid may be a pentacyclic triterpenoid, such as oleanolic acid and hederagenin, or a tetracyclic triterpenoid compound, such as cycloastragenol and elemadienonic acid.

Accordingly, in a first aspect of the invention, there is provided a composition comprising:
(a) a triterpenoid compound according to formula (I), or a pharmaceutically or cosmetically acceptable salt, prodrug, solvate, tautomer or stereoisomer thereof; and
(b) a plant extract selected from extracts of plants belonging to the genus Garcinia, Rubus, Fragaria, Ilex and Hedera,

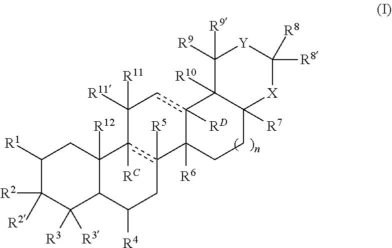

wherein:
—Y— is a covalent bond and —X— is —CH₂—, —CH₂CH₂—, or —X— is absent, and the carbon atoms to which —X— is connected are bonded to H,
—Y— is —O—, and —X— is absent, and the carbon atoms to which —X— is connected are bonded to H,
a dashed bond indicates the presence of a double or single bond between the ring carbon atoms, where only one double bond is present within the ring,
R¹, R³, R^{3′}, R⁴, R⁵, R⁶, R⁷, R⁸, R^{8′}, R⁹, R^{9′}, R¹⁰, and R¹² are independently selected from H, F, OH, SH, C₁₋₆ alkyl, C₁₋₄ haloalkyl, C₁₋₄ hydroxyalkyl, C₂₋₆ alkenyl, OR¹³, CO₂R¹⁴, OC(═O)R¹⁵ and, where —Y— is —O— and —X— is absent, R⁸ and R⁹ may together form a heterocycle, and R⁵ is absent when the ring carbon to which it is connected is in a double bond;
R² and R^{2′} are independently selected from H, F, OH, SH, C₁₋₆ alkyl, C₁₋₄ haloalkyl, C₂₋₆ alkenyl, OR¹⁶, CO₂R¹⁷, OC(═O)R¹⁸, or R² and R^{2′} together represent ═O;
R¹¹ and R^{11′} are independently selected from H, F, OH, SH, C₁₋₆ alkyl, I, C₁₋₄haloalkyl, C₂₋₆ alkenyl, OR¹⁹, CO₂R²⁰, OC(═O)R²¹, or R¹¹ and R^{11′} together represent ═O;
R^{C} is absent, or where the ring carbon to which it is connected is not in a double bond, R^{C} is H, F, OH, SH, C₁₋₆ alkyl, C₁₋₄ haloalkyl, C₁₋₄ hydroxyalkyl, C₂₋₆ alkenyl, OR¹³, CO₂R¹⁴, OC(═O)R¹⁵, or together with R¹² may form a carbocycle;
R^{D} is absent, or where the ring carbon to which it is connected is not in a double bond, R^{D} is H, F, OH, SH, C₁₋₆ alkyl, C₁₋₄ haloalkyl, C₁₋₄ hydroxyalkyl, C₂₋₆ alkenyl, OR¹³, CO₂R¹⁴, OC(═O)R¹⁵, R¹³, R¹⁴, R¹⁵, R¹⁶, R¹⁷, R¹⁸ R¹⁹, R²⁰, and R²¹ are independently selected from H, C₁₋₆ alkyl and C₁₋₄ haloalkyl; and
n is 0 or 1.

Preferred features of the composition, such as the triterpenoid compound and the plant extract, are set out below.

The inventors have found that compositions of the first aspect reduce the production of lipids in sebocytes.

Accordingly, in a second aspect of the invention, there is provided a method for decreasing lipid production in sebocytes, the method comprising contacting the sebocytes with a composition of the first aspect. This method may be in vivo or ex vivo, such as in vitro.

The inventors have found that the compositions of the first aspect may reduce or ameliorate cosmetic problems associated with over-production of lipids in the skin, such as oily or shiny skin, oily hair, enlarged skin pores, undesirable body odour, and decreased retention of make-up products on the skin.

Accordingly, in a third aspect of the invention, there is provided a cosmetic method for decreasing lipid production in the skin of an individual, the method comprising contacting the skin with a composition of the first aspect.

The inventors have found that the compositions of the first aspect are useful in the treatment or prophylaxis of medical problems associated with over-production of lipids in the skin, such as acne vulgaris and rosacea.

Accordingly, in a fourth aspect of the invention, there is provided a composition of the first aspect for use in a method of treatment.

In a further related aspect of the invention, there is provided a composition of the first aspect for use in a method of treating a skin disease or disorder associated with over-production of lipids in the skin, such as acne vulgaris and rosacea.

These and other aspects and embodiments of the invention are described in further detail below.

### SUMMARY OF THE FIGURES

The present invention is described with reference to the figures listed below.

FIG. 1 shows mean fluorescence intensity obtained by flow cytometry of sebocytes cells treated with AdipoRed™ dye. Cells were incubated with test compositions for 3 days. From left to right, DMSO (negative control), EGCG (positive control), salicylic acid (positive control), oleanolic acid, Rubus idaeus extract, and a combination of oleanolic acid and Rubus idaeus extract according to an embodiment of the invention.

FIG. 2 shows mean fluorescence intensity obtained by flow cytometry of sebocytes cells treated with AdipoRed™ dye. Cells were incubated with test compositions for 3 days. From left to right, DMSO (negative control), EGCG (positive control), salicylic acid (positive control), oleanolic acid, Hedera helix extract, and a combination of oleanolic acid and Hedera helix extract according to an embodiment of the invention.

FIG. 3 shows mean fluorescence intensity obtained by flow cytometry of sebocytes cells treated with AdipoRed™ dye. Cells were incubated with test compositions for 3 days. From left to right, DMSO (negative control), EGCG (positive control), salicylic acid (positive control), oleanolic acid, Ilex aquifolium extract, and a combination of oleanolic acid and Ilex aquifolium extract according to an embodiment of the invention.

FIG. 4 shows mean fluorescence intensity obtained by flow cytometry of sebocytes cells treated with AdipoRed™ dye. Cells were incubated with test compositions for 3 days. From left to right, DMSO (negative control), EGCG (positive control), oleanolic acid, Garcinia mangostana extract, and a combination of oleanolic acid and Garcinia mangostana extract according to an embodiment of the invention.

FIG. 5 shows mean fluorescence intensity obtained by flow cytometry of sebocytes cells treated with AdipoRed™ dye. Cells were incubated with test compositions for 3 days. From left to right, DMSO (negative control), EGCG (positive control), beta-elemonic, Rubus idaeus extract, and a combination of beta-elemonic and Rubus Idaeus extract according to an embodiment of the invention.

FIG. 6 shows mean fluorescence intensity obtained by flow cytometry of sebocytes cells treated with AdipoRed™ dye. Cells were incubated with test compositions for 3 days. From left to right, DMSO (negative control), EGCG (positive control), cycloastragenol, Rubus idaeus extract, and a combination of cycloastragenol and Rubus idaeus extract according to an embodiment of the invention.

FIG. 7 shows mean fluorescence intensity obtained by flow cytometry of sebocytes cells treated with AdipoRed™ dye. Cells were incubated with test compositions for 3 days. From left to right, DMSO (negative control), EGCG (positive control), oleanolic acid, Fragaria vesca extract, and a combination of oleanolic acid and Fragaria vesca extract according to an embodiment of the invention.

Within the figures the following labels are used with respect to the comparison in a one-way analysis of variance (one-way ANOVA):
*P value: <0.05 (Statistically significant compared to DMSO)
**P value: <0.01 (Statistically significant compared to DMSO)
***P value: 0.001 (Statistically significant compared to DMSO)
****P value: <0.0001 (Statistically significant compared to DMSO)
#P value: <0.05 (Statistically significant compared to Oleanolic Acid)
##P value: <0.01 (Statistically significant compared to Oleanolic Acid)
###P value: <0.001 (Statistically significant compared to Oleanolic Acid)
####P value: <0.001 (Statistically significant compared to Oleanolic Acid)

### DETAILED DESCRIPTION OF THE INVENTION

The invention relates to compositions comprising a plant extracts in combination with triterpenoid compounds, and the use of the composition in a method for decreasing lipid production in sebocytes. Also provided is the use of the composition in cosmetic method for decreasing lipid production in the skin of an individual, and the use of the composition in a method of treating a skin disease or disorder related to increased lipid production in sebocytes.

The following preferences may apply to all aspects of the invention as described above. The preferences may be combined in any combination.

### Triterpenoid Compounds

The composition of the invention contains a triterpenoid together with a plant extract. The triterpenoid may be a pentacyclic triterpenoid, such as oleanolic acid and hederagenin, or a tetracyclic triterpenoid compound, such as cycloastragenol and elemadienonic acid.

A pentacyclic triterpenoid contains five fused carbocyclic rings, and these rings are substituted. A tetracyclic triterpenoid contains four fused carbocyclic rings, and these rings are substituted.

One of the fused carbocyclic rings in the pentacyclic or tetracyclic triterpenoid may contain an endo carbon-carbon-double bond. Thus, there is a double bond within a ring, between neighbouring carbon ring atoms. In one embodiment the fused rings are saturated.

In one embodiment, the triterpenoid compound is a triterpenoid that is not present within, or otherwise derived from, the plant extract with which it is used in combination.

The composition of the invention comprises a triterpenoid compound according to formula (I):

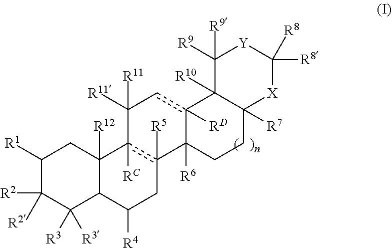

where:
—Y— is a covalent bond and —X— is —CH₂—, —CH₂CH₂—, or —X— is absent, and the carbon atoms to which —X— is connected are bonded to H, or
—Y— is —O—, and —X— is absent, and the carbon atoms to which it is connected are bonded to H,
a dashed bond indicates the presence of a double or single bond between the ring carbon atoms, where only one double bond is present within the ring,
R¹, R³, R^{3′}, R⁴, R⁵, R⁶, R⁷, R⁸, R^{8′}, R⁹, R^{9′}, R¹⁰, and R¹² are independently selected from H, F, OH, SH, C₁₋₆ alkyl, C₁₋₄ haloalkyl, C₁₋₄ hydroxyalkyl, C₂₋₆ alkenyl, OR¹—, CO₂R¹⁴, OC(═O)R¹⁵ and, where —Y— is —O— and —X— is absent, R⁸ and R⁹ may together form a heterocycle, and R⁵ is absent when the ring carbon to which it is connected is in a double bond;
R² and R^{2′} are independently selected from H, F, OH, SH, C₁₋₆ alkyl, C₁₋₄ haloalkyl, C₂₋₆ alkenyl, OR¹⁶, CO₂R¹⁷, OC(═O)R¹⁸, or R² and R^{2′} together represent ═O;
R¹¹ and R^{11′} are independently selected from H, F, OH, SH, C₁₋₆ alkyl, I, C₁₋₄haloalkyl, C₂₋₆ alkenyl, OR¹⁹, CO₂R²⁰, OC(═O)R²¹, or R¹¹ and R^{11′} together represent ═O;
R^{C} is absent, or where the ring carbon to which it is connected is not in a double bond, R^{C} is H, F, OH, SH, C₁₋₆ alkyl, C₁₋₄ haloalkyl, C₁₋₄ hydroxyalkyl, C₂₋₆ alkenyl, OR¹³, CO₂R¹⁴, OC(═O)R¹⁵, or together with R¹² may form a carbocycle;
R^{D} is absent, or where the ring carbon to which it is connected is not in a double bond, R^{D} is H, F, OH, SH, C₁₋₆ alkyl, C₁₋₄ haloalkyl, C₁₋₄ hydroxyalkyl, C₂₋₆ alkenyl, OR¹³, CO₂R¹⁴, OC(═O)R¹⁵,
R¹³, R¹⁴, R¹⁵, R¹⁶, R¹⁷, R¹⁸ R¹⁹, R²⁰, and R²¹ are independently selected from H, C₁₋₆ alkyl and C₁₋₄ haloalkyl; and
n is 0 or 1.

An alkyl group is monovalent saturated hydrocarbon group. The alkyl group may be a C₁₋₆ alkyl group, for example a C₁₋₄, C₁₋₃ or a C₁₋₂ alkyl group, such as C₁ alkyl (methyl). In this context, the prefix (e.g. C₁₋₆) denotes the number of carbon atoms in the hydrocarbon backbone. The alkyl group may be linear or branched.

Examples of C₁₋₆ linear alkyl groups include methyl (-Me), ethyl (-Et), n-propyl (-nPr), n-butyl (-nBu), n-pentyl (-Amyl) and n-hexyl.

Examples of C₁₋₆ branched alkyl groups include iso-propyl (-iPr), iso-butyl (-iBu), sec-butyl (-sBu), tert-butyl (-tBu), iso-pentyl, sec-pentyl, tert-pentyl, neo-pentyl, iso-hexyl, sec-hexyl, tert-hexyl and neo-hexyl.

A haloalkyl group is an alkyl group in which one or more hydrogen atoms, such as one or all of the hydrogen atoms, is replaced with a halogen atom, for example F, Cl, Br and I. The haloalkyl group may be a C₁₋₄ haloalkyl group, for example a C₁₋₃ or a C₁₋₂ haloalkyl group, and these may be monohalo or perhalo alkyl groups. In this context, the prefix (e.g. C₁₋₄) denotes the number of carbon atoms in the hydrocarbon backbone.

Examples of C₁₋₄ haloalkyl groups include chloromethyl (—CH₂Cl), fluoromethyl (—CH₂F), difluoromethyl (—CHF₂), trifluoromethyl (—CF₃), chloroethyl (—C₂H₄Cl), monolfluorethyl (—C₂H₄F), pentafluoroethyl (—C₂F₃), heptafluoropropyl (—C₃F₇) and nonafluorobutyl (—C₄F₉).

A hydroxyalkyl group is an alkyl group in which one or more hydrogen atoms, such as on hydrogen atom, is replaced with hydroxy (—OH). The hydroxyalkyl group may be a C₁₋₄ hydroxyalkyl group, for example a C₁₋₃ or a C₁₋₂ hydroxyalkyl group, such as a C₁ hydroxyalkyl group. A hydroxyalkyl group may be a monohydroxyalkyl group. In this context, the prefix (e.g. C₁₋₄) denotes the number of carbon atoms in the hydrocarbon backbone.

Examples of C₁₋₄ hydroxyalkyl groups include hydroxymethyl (—CH₂OH) and hydroxyethyl (such as —C₂H₄OH).

An alkenyl group is a monovalent unsaturated hydrocarbon group containing one or more carbon-carbon double bonds. The alkenyl group may be a C₂₋₆ alkenyl group, for example a C₂₋₅, C₂₋₄ or a C₂₋₃ alkenyl group. In this context, the prefix (e.g. C₂₋₆) denotes the number of carbon atoms in the hydrocarbon backbone. The alkenyl group may be linear or branched. The alkenyl group may contain one or more, such as two or more, such as three more, carbon-carbon double bonds. Where two or more double bonds are present, these may be conjugated or not.

Examples of C₂₋₆ linear alkenyl groups include ethenyl (vinyl), 1-propenyl, 2-propenyl (allyl), 1-butenyl, 2-butenyl, 1-pentenyl, 2-pentenyl, 3-pentenyl, 1-hexenyl, and 2-hexenyl, 3-hexenyl, and 4-hexenyl.

Examples of C₂₋₆ branched alkenyl groups include isopropenyl (1-methylvinyl), isobutenyl (2-methyl-1-propenyl), 1-isopentenyl, (3-methyl-1-butenyl), and 2-isopentenyl (3-methyl-2-butenyl).

In a preferred embodiment, R¹, R³, R^{3′}, R⁴, R⁵, R⁶, R⁷, R⁸, R^{8′}, R⁹, R^{9′}, R¹⁰, and R¹² are not all H.

In a preferred embodiment, R¹, R³, R^{3′}, R⁴, R⁵, R⁶, R⁷, R⁸, R^{8′}, R⁹, R^{9′}, R¹⁰, and R¹² are not all OH.

In a preferred embodiment, R¹, R³, R^{3′}, R⁴, R⁵, R⁶, R⁷, R⁸, R^{8′}, R⁹, R^{9′}, R¹⁰, and R¹² are not all CO₂H.

In a preferred embodiment, at least four groups selected from R¹, R³, R^{3′}, R⁴, R⁵, R⁶, R⁷, R⁸, R^{8′}, R⁹, R^{9′}, R¹⁰, and R¹² are H.

In a preferred embodiment:
R¹, R³, R^{3′}, R⁴, R⁵, R⁶, R⁷, R⁸, R^{8′}, R⁹, R^{9′}, R¹⁰, and R¹² are independently selected from H, F, OH, SH, C₁₋₆ alkyl, C₁₋₄ fluoroalkyl, C₂₋₆ alkenyl, OR¹³, CO₂R¹⁴, OC(═O)R¹⁵;
R² and R^{2′} are independently selected from H, F, OH, SH, C₁₋₆ alkyl, C₁₋₄ fluoroalkyl, C₂₋₆ alkenyl, OR¹⁶, CO₂R¹⁷, OC(═O)R¹⁸, or R² and R^{2′} together represent ═O;
R¹¹ and R^{11′} are independently selected from H, F, OH, SH, C₁₋₆ alkyl, I, C₁₋₄ fluoroalkyl, C₂₋₆ alkenyl, OR¹⁹, CO₂R²⁰, OC(═O)R²¹, or R¹¹ and R^{11′} together represent ═O;
R¹³, R¹⁴, R¹⁵, R¹⁶, R¹⁷, R¹⁸, R¹⁹, R²⁰, and R²¹ are independently selected from H, C₁₋₆ alkyl and C₁₋₄ fluoroalkyl; and
n is 1 or 2.

A fluoroalkyl group is an alkyl group in which one or more hydrogen atoms is replaced with a fluorine atom, i.e. F. The fluoroalkyl group may be a C₁₋₄ fluoroalkyl group, for example a C₁₋₃ or a C₁₋₂ fluoroalkyl group. In this context, the prefix (e.g. C₁₋₄) denotes the number of carbon atoms in the hydrocarbon backbone.

Examples of C₁₋₄ fluoroalkyl groups include monofluoromethyl (—CH₂F), difluoromethyl (—CHF₂), trifluoromethyl (—CF₃), pentafluoroethyl (—C₂F₃), heptafluoropropyl (—C₃F₇) and nonafluorobutyl (—C₄F₉).

The Groups —Y— and —X—

In one embodiment —Y— is a covalent bond and —X— is —CH₂— or —CH₂CH₂—.

When —Y— is a covalent bond and —X— is —CH₂— a cyclopentenyl ring is formed.

When —Y— is a covalent bond and —X— is —CH₂CH₂— a cyclohexyl ring is formed.

In one embodiment —Y— is a covalent bond and —X— is absent, and the carbon atoms to which —X— is connected are bonded to H.

In a preferred embodiment, —Y— is a covalent bond and —X— is —CH₂CH₂—. Accordingly, the compound of formula (I) is:

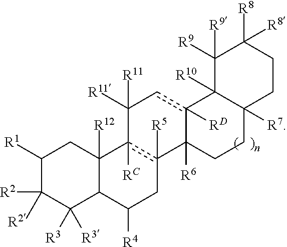

In a preferred embodiment, —Y— is a covalent bond and —X— is —CH₂—. Accordingly, the compound of formula (I) is:

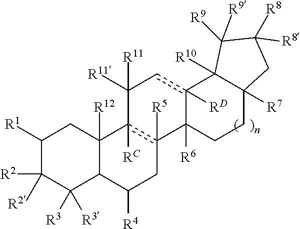

In one embodiment, —Y— is —O—, and —X— is absent, and the carbon atoms to which —X— is connected are bonded to H.

When —X— is absent, and the carbon atoms to which —X— is connected are bonded to H, no ring is formed.

Double Bonds and R^{C} and R^{D}

In one embodiment, the compound of formula (I) is:

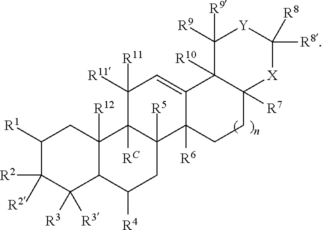

In one embodiment, the compound of formula (I) is:

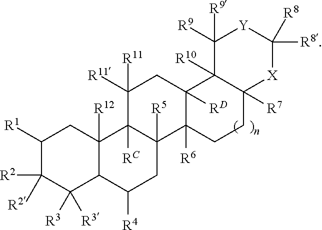

In one embodiment, the compound of formula (I) is:

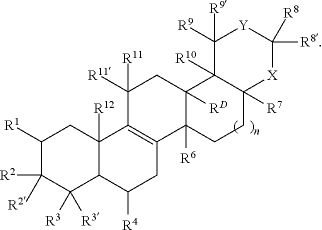

Where R^{C} is present, in one embodiment it may be H.

In one embodiment, where R^{C} is present it may together with R¹², and the carbon ring atoms to which they are attached, form a carbocycle, such as a C₃₋₆ carbocycle. This carbocycle may be a cyclopropane ring.

Where R^{D} is present, in one embodiment it may be H or C₁₋₆ alkyl.

### Value of n

In one embodiment, n is 0. The ring is therefore a cyclopentyl ring. This is preferred where —X— is absent, and the carbon atoms to which —X— is connected are bonded to H In one embodiment, n is 1. The ring is therefore a cyclohexyl ring. This is preferred where —X— is —CH₂—, —CH₂CH₂—.

The Groups R¹, R³, R^{3′}, R⁴, R⁵, R⁶, R⁷, R⁸, R^{8′}, R⁹, R^{9′}, R¹⁰, and R¹²

In a preferred embodiment:
R¹, R³, R^{3′}, R⁴, R⁵, R⁶, R⁷, R⁸, R^{8′}, R⁹, R^{9′}, R¹⁰, and R¹² are independently selected from H, F, OH, SH, OR¹³, CO₂R¹⁴, OC(═O)R¹⁵, C₁₋₆ alkyl, CF₃, C₂F₅ and C₂₋₆ alkenyl; and
R¹³, R¹⁴, and R¹⁵ are independently selected from H, C₁₋₆ alkyl, CF₃, and C₂F₅.

In a preferred embodiment:
R¹, R³, R^{3′}, R⁴, R⁵, R⁶, R⁷, R⁸, R^{8′}, R⁹, R^{9′}, R¹⁰, and R¹² are independently selected from H, F, OH, SH, OR¹³, CO₂R⁴, OC(═O)R¹⁵, C₁₋₆ alkyl, CF₃, and C₂₋₆ alkenyl; and
R¹³, R¹⁴, and R¹⁵ are independently selected from H, C₁₋₆ alkyl, and CF₃.

In a preferred embodiment:
R¹, R³, R^{3′}, R⁴, R⁵, R⁶, R⁷, R⁸, R^{8′}, R⁹, R^{9′}, R¹⁰, and R¹² are independently selected from H, F, OH, OR¹³, COR¹⁴, OC(═O)R¹⁵, C₁₋₆ alkyl, and C₂₋₆ alkenyl; and
R¹³, R¹⁴, and R¹⁵ are independently selected from H and C₁₋₆ alkyl.

In a preferred embodiment:
R¹, R³, R^{3′}, R⁴, R⁵, R⁶, R⁷, R⁸, R^{8′}, R⁹, R^{9′}, R¹⁰, and R¹² are independently selected from H, OH, Me, Et, vinyl, allyl, 1-methylvinyl, OMe, OEt, CH₂OH, CH₂CH₂OH, CO₂H, CO₂Me, and OC(O)Me.

In a preferred embodiment R⁴ is H or OH.

Where —Y— is —O— and —X— is absent, R⁸ and R⁹ may together with the carbon atoms to which the are attached form a heterocycle. The heterocycle may be a tetrahydrofuran or tetrahydropyran. For example, cycloastragenol is a compound having a tetrahydrofuran group within the triterpenoid.

In a preferred embodiment:
R¹ and/or R⁴ and/or R¹⁰ are H.

In a preferred embodiment:
R⁵ and/or R⁶ and/or R¹² are Me.

In a preferred embodiment:
R⁷ is CO₂H.
   The Groups R² and R^{2′}

In a preferred embodiment:
R² and R^{2′} are independently selected from H, OH, OMe, OEt, C₁₋₄ alkyl, C₂₋₄ alkenyl, CO₂H, CO₂Me, CO₂Et, OC(O)Me, and OC(O)Et, or R² and R^{2′} together represent ═O.

In a preferred embodiment:
R² and R^{2′} are independently selected from H, OH, Me, Et, vinyl, allyl, 1-methylvinyl, OMe, CO₂H, CO₂Me, and OC(O)Me, or R² and R^{2′} together represent ═O.
   The Groups R¹¹ and R^{11′}

In a preferred embodiment:
R¹¹ and R^{11′} are independently selected from H, OH, OMe, OEt, C₁₋₄ alkyl, C₂₋₄ alkenyl, CO₂H, CO₂Me, CO₂Et, OC(O)Me, and OC(O)Et, or R² and R^{2′} together represent ═O.

In a preferred embodiment:
R¹¹ and R^{11′} are independently selected from H, OH, Me, Et, vinyl, allyl, 1-methylvinyl, OMe, CO₂H, CO₂Me, and OC(O)Me, or R² and R^{2′} together represent ═O.

### Preferred Formula

In a particularly preferred embodiment, the triterpenoid compound is a compound of formula (IIa):

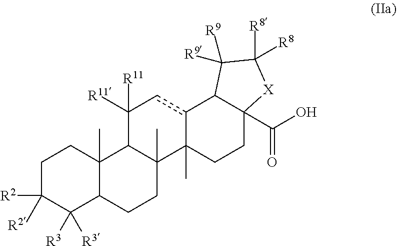

wherein:
R³, R^{3′}, R⁸, R^{8′}, R⁹, and R^{9′} are independently selected from H, OH, Me, Et, vinyl, allyl, 1-methylvinyl, OMe, OEt, CH₂OH, CH₂CH₂OH, CO₂H, CO₂Me, and OC(O)Me;
R² and R^{2′} are independently selected from H, OH, Me, Et, vinyl, allyl, 1-methylvinyl, OMe, OEt, CH₂OH, CH₂CH₂OH, CO₂H, CO₂Me, and OC(O)Me, or R^{2A} and R^{2A′} together represent ═O;
R¹¹ and R^{11′} are independently selected from H, OH, Me, Et, vinyl, allyl, 1-methylvinyl, OMe, OEt, CH₂OH, CH₂CH₂OH, CO₂H, CO₂Me, and OC(O)Me, or R¹¹ and R^{11′} together represent ═O;
X is —CH₂—, —CH₂CH₂—; and
the dashed bond indicates the presence of a double or single bond between the ring carbon atoms.

Within formula (IIa), where the dashed bond indicates a single bond, it is understood that the carbon ring atoms are connected to H.

In one embodiment, a double bond is present between the ring carbon atoms.

In one embodiment, —X— is —CH₂CH₂—.

In one embodiment, —X— is —CH₂—.

In a preferred embodiment:
R¹, R³, R⁶, and R⁷ are independently selected from H, OH, OR¹³, CO₂R¹⁴, OC(═O)R¹⁵, Me, Et, and C₁₋₄ alkenyl; and
R¹³, R¹⁴ and R¹⁵ are independently selected from H, Me and Et.

In a preferred embodiment:
R³, R^{3′}, R⁸, R^{8′} R¹¹ and R^{1A′} are independently selected from H, OH, Me, OMe, CH₂OH, and CO₂H;
R² and R^{A′} are independently selected from H, OH, Me, OMe, CH₂OH, CO₂H and OC(O)Me, or R² and R^{2′} together represent ═O;
R⁹ and R^{9′} H, OH, Me, 1-methylvinyl, OMe, CH₂OH, and CO₂H.

In a particularly preferred embodiment, the triterpenoid compound is selected from a compound of formulae (II) to (VII):

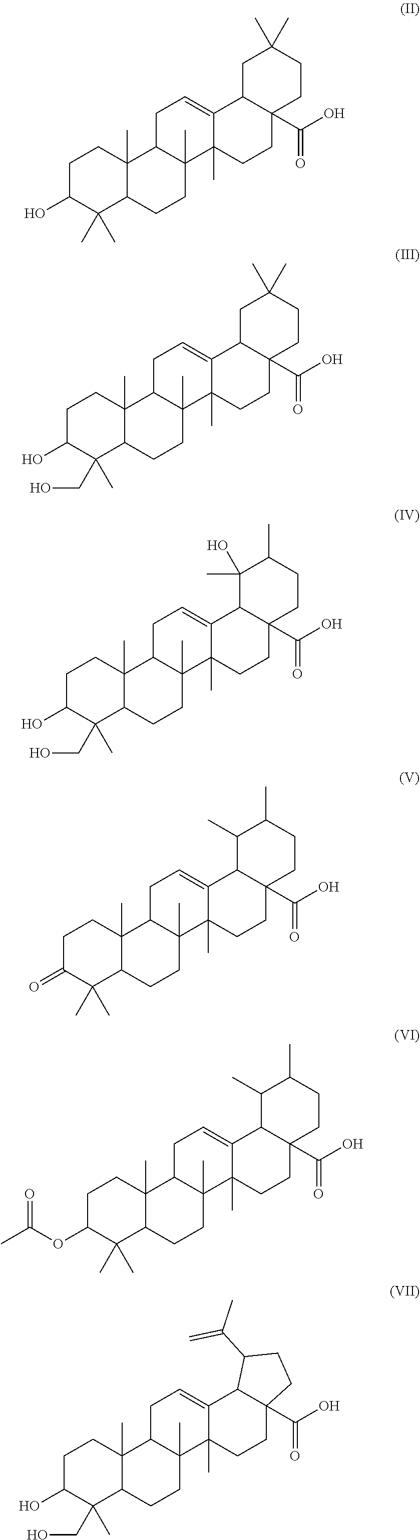

In another embodiment, the triterpenoid compound is selected from a compound of formula (VIII):

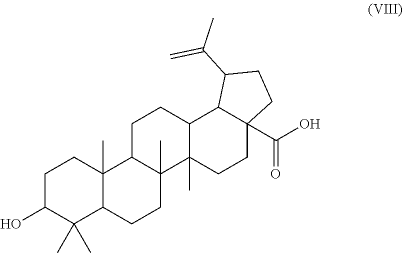

In a particularly preferred embodiment, the triterpenoid compound is a compound of formula (IIb):

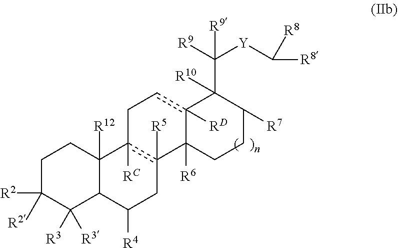

wherein:
Y— is a covalent bond or —O—,
a dashed bond indicates the presence of a double or single bond between the ring carbon atoms, where only one double bond is present within the ring,
R³, R^{3′}, R⁴, R⁵, R⁶, R⁷, R⁸, R^{8′}, R⁹, R^{9′}, R¹⁰, and R¹² are independently selected from H, F, OH, SH, C₁₋₆ alkyl, C₁₋₄haloalkyl, C₁₋₄ hydroxyalkyl, C₂₋₆ alkenyl, OR¹³, CO₂R¹⁴, OC(═O)R¹⁵ and, where —Y— is —O— and —X— is absent, R⁸ and R⁹ may together form a heterocycle, and R⁵ is absent when the ring carbon to which it is connected is in a double bond;
R² and R^{2′} are independently selected from H, F, OH, SH, C₁₋₆ alkyl, C₁₋₄ haloalkyl, C₂₋₆ alkenyl, OR¹⁶, CO₂R¹⁷, OC(═O)R¹⁸, or R² and R^{2′} together represent ═O;
R^{C} is absent, or where the ring carbon to which it is connected is not in a double bond, R^{C} is H, F, OH, SH, C₁₋₆ alkyl, C₁₋₄ haloalkyl, C₁₋₄ hydroxyalkyl, C₂₋₆ alkenyl, OR¹³, CO₂R¹⁴, OC(═O)R¹⁵, or together with R¹² may form a carbocycle;
R^{D} is absent, or where the ring carbon to which it is connected is not in a double bond, R^{D} is H, F, OH, SH, C₁, alkyl, C₁₋₄ haloalkyl, C₁₋₄ hydroxyalkyl, C₂₋₆ alkenyl, OR¹³, CO₂R¹⁴, OC(═O)R¹⁵, R¹³, R¹⁴, R¹⁵, R¹⁶, R¹⁷, R¹⁸ R¹⁹, R²⁰, and R²¹ are independently selected from H, C₁₋₆ alkyl and C₁₋₄ haloalkyl; and
n is 0 or 1.

In a preferred embodiment:
R¹, R³, R⁶, and R⁷ are independently selected from H, OH, OR¹³, CO₂R¹⁴, OC(═O)R¹⁵, Me, Et, and C₁₋₄ alkenyl; and
R¹³, R¹⁴ and R¹⁵ are independently selected from H, Me and Et.

In a preferred embodiment:
R³, R^{3′}, R⁸, R^{8′} R¹¹ and R^{11′} are independently selected from H, OH, Me, OMe, CH₂OH, and CO₂H;
R² and R^{2′} are independently selected from H, OH, Me, OMe, CH₂OH, CO₂H and OC(O)Me, or R² and R^{2′} together represent ═O;
R⁹ and R^{9′} H, OH, Me, 1-methylvinyl, OMe, CH₂OH, and CO₂H.

In another embodiment, the triterpenoid compound is selected from a compound of formulae (IX) and (X):

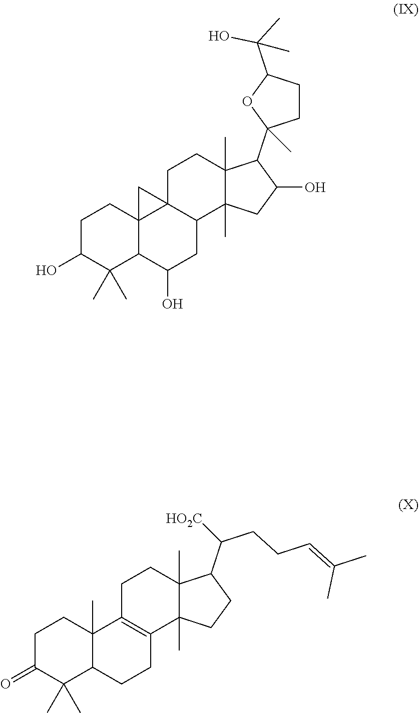 [SMILES: CC(C)(O)C1CCC(C)(C2C(O)CC3(C)C4CC(O)C5C(C)(C)C(O)CCC56CC46CCC23C)O1 ; CC(C)=CCCC(C(=O)O)C1CCC2(C)C3=C(CCC12C)C1(C)CCC(=O)C(C)(C)C1CC3]

### Preferred Compounds

In a particularly preferred embodiment, the triterpenoid compound is selected from oleanolic acid, hederagenin, rotundic acid, ursonic acid, acetylursolic acid, anemosapogenin, betulinic acid (lupatic acid), cycloastragenol, and β-elemonic acid (elemadienonic acid).

The triterpenoid compound may be a pentacyclic triterpenoid.

The triterpenoid compound may be a tetracyclic triterpenoid.

The triterpenoid compound may be selected from oleanolic acid, betulinic acid, cycloastragenol, and β-elemonic acid.

The triterpenoid compound may be selected from oleanolic acid, cycloastragenol, and β-elemonic acid.

The triterpenoid compound may be selected from oleanolic acid and betulinic acid.

The triterpenoid compound may be selected from oleanolic acid, β-elemonic acid and cycloastragenol.

The triterpenoid compound may be oleanolic acid.

The triterpenoid compound may be β-elemonic acid.

The triterpenoid compound may be β-elemonic acid.

The triterpenoid compounds described herein, such as the triterpenoid compounds of formula (I), may be provided in free base form. Alternatively, the triterpenoid compounds may be provided in the form of a salt, such as a pharmaceutically or cosmetically acceptable salt. Examples of pharmaceutically acceptable salts are discussed in Berge et al., 1977, “Pharmaceutically Acceptable Salts,” J. Pharm. Sci., Vol. 66, pp. 1-19.

In some embodiments, the triterpenoid compounds described herein are provided in a protonated form together with a suitable counter anion.

Suitable counter anions include both organic and inorganic anions. Example of inorganic anions include those derived from inorganic acids, including chloride (Cl⁻), bromide (Br⁻), iodide (I⁻), sulfate (SO₄²⁻), sulfite (SO₃²⁻), nitrate (NO₃⁻), nitrite (NO₂⁻), phosphate (PO₄³⁻), and phosphite (PO₃³⁻). Examples of organic anions include 2-acetoxybenzoate, acetate, ascorbate, aspartate, benzoate, camphorsulfonate, cinnamate, citrate, edetate, ethanedisulfonate, ethanesulfonate, formate, fumarate, gluconate, glutamate, glycolate, hydroxymalate, carboxylate, lactate, laurate, lactate, maleate, malate, methanesulfonate, oleate, oxalate, palmitate, phenylacetate, phenylsulfonate, propionate, pyruvate, salicylate, stearate, succinate, sulfanilate, tartarate, toluenesulfonate, and valerate. Examples of suitable polymeric organic anions include those derived from tannic acid and carboxymethyl cellulose.

In some embodiments, the triterpenoid compounds disclosed herein are provided in a deprotonated form together with a suitable counter cation.

Suitable counter cations include both organic and inorganic cations. Suitable counterions include both inorganic and organic cations. Examples of suitable inorganic cations include alkali metal ions such as Na⁺ and K⁺, alkaline earth cations such as Ca²⁺ and Mg²⁺, and other cations such as Al³⁺. Examples of suitable organic cations include the ammonium ion (i.e., NH₄⁺) and substituted ammonium ions (e.g., NH₃R⁺, NH₂R²⁺, NHR₃⁺, NR₄⁺). Examples of substituted ammonium ions include those derived from ethylamine, diethylamine, dicyclohexylamine, triethylamine, butylamine, ethylenediamine, ethanolamine, diethanolamine, piperazine, benzylamine, phenylbenzylamine, choline, meglumine, and tromethamine, as well as amino acids, such as lysine and arginine. An example of a common quaternary ammonium ion is N(CH₃)₄⁺.

Unless otherwise specified, a reference to a particular compound also includes salt forms thereof.

The triterpenoid compounds described herein may be provided in the form of a solvate (a complex of solute (e.g., compound, salt of compound) and solvent). Examples of solvates include hydrates, for example, a mono-hydrate, a di-hydrate and a tri-hydrate.

The triterpenoid compounds described herein may be provided in desolvated form, for example, in dehydrated form.

The composition of the invention may comprise an effective amount, such as a therapeutically or cosmetically effective amount, of the triterpenoid compound described herein, such as the triterpenoid compounds of formula (I).

In some embodiments, the composition of the invention comprises the triterpenoid compound in an amount of from 0.0005 wt % to 10 wt %, such as based on the total weight of the composition. In a preferred embodiment, the composition of the invention comprises the triterpenoid compound in an amount of from 0.1 wt % to 5 wt %, such as 0.1 wt % to 3 wt %, and such as 0.2 wt % to 3 wt %, and more preferably 0.2 wt % to 2 wt %.

The triterpenoid compound may be present in an amount that is at most 2 wt %, 3 wt %, 5 wt %., or 10 wt %.

The triterpenoid compound may be present in an amount that is at least 0.0005 wt %, 0.001 wt %, 0.005 wt %, 0.01 wt %, 0.05 wt %, 0.1 wt %, 0.5 wt %, or 1.0 wt %.

### Plant Extracts

The composition of the invention comprises a plant extract. The plant extract is selected from extracts of plants belonging to the genus Garcinia, Rubus, Fragaria, Ilex and Hedera.

The plant extract may be derived from any suitable species of the genus Garcinia, Rubus, Fragaria, Ilex and Hedera.

Suitable plant extracts are commercially available and can be purchased from, for example, BOCSCI (New York, USA).

Suitable species of plants belonging to the genus Garcinia include Garcinia mangostana (mangosteen).

Suitable species of plants belonging to the genus Rubus include Rubus idaeus (red raspberry). Rubus idaeus is widely distributed throughout Europe and is native to, for example, the United Kingdom.

Suitable species of plants belonging to the genus Hedera include Hedera helix (common ivy). Hedera helix is widely distributed throughout Europe and is native to, for example, the United Kingdom.

Suitable species of plants belonging to the genus Ilex include Ilex aquifolium (common holly). Ilex aquifolium is widely distributed throughout Europe and is native to, for example, the United Kingdom.

Suitable species of plants belonging to the genus Fragaria include Fragaria vesca (wild strawberry) and Fragaria×ananassa (garden strawberry). Fragaria vesca and is widely distributed throughout Europe and is native to, for example, the United Kingdom. Fragaria×ananassa is widely cultivated across Europe, including the United Kingdom.

The plant extract may be derived from any suitable part of the plant. Suitable parts of the plant include roots, stems, leaves, flowers, fruits and seeds.

Examples of suitable fruit extracts include Rubus idaeus fruit extract, Garcinia mangostana pericarp extract and Fragaria vesca fruit extract.

Examples of suitable leaf extracts include Ilex aquifolium leaf extract.

The plant extract may be prepared using any suitable extraction method. Suitable extraction methods include extraction using an organic solvent and extraction using an aqueous solvent (aqueous extraction).

Typically, the plant extract is an aqueous extract.

In a further embodiment, the plant extract is selected from Rubus idaeus extract, Garcinia mangostana extract, Hedera helix extract, Ilex aquifolium extract and Fragaria vesca extract.

In a preferred embodiment, the plant extract is selected from extracts of plants belonging to the genus Rubus and Fragaria. In a preferred embodiment, the plant extract is selected from Rubus idaeus extract and Fragaria vesca extract.

In a preferred embodiment, the plant extract is selected from extracts of plants belonging to the genus Garcinia. In a preferred embodiment, the plant extract is Garcinia mangostana extract.

In a preferred embodiment, the plant extract is selected from extracts of plants belonging to the genus Hedera and Ilex. In a preferred embodiment, the plant extract is selected from Hedera helix extract and Ilex aquifolium extract.

In a preferred embodiment, the plant extract is Rubus Idaeus extract.

In another embodiment, the plant extract is Hedera helix extract.

In a further embodiment, the plant extract is Ilex aquifolium leaf extract.

In one embodiment, the plant extract is Garcinia mangostana extract.

In yet a further embodiment, the plan extract is Fragaria vesca extract.

A triterpenoid compound may not be present within, or otherwise derived from, the plant extract with which it is used in combination.

The composition of the invention may comprise an effective amount, such as a therapeutically or cosmetically effective amount, of the plant extract described herein.

A Rubus extract, such as a Rubus Idaeus extract, may comprise one or more of, such as all of, the following compounds given in Table 1, at an amount in the range given. The extract here may be a dry extract.

TABLE 1
Components of Rubus idaeus solid plant extract

| Compound | Concentration Range (μg/g = ppm) |
|---|---|
| Adenine | 0.7-7.1 |
| Betaine | 1.2-3.4 |
| Choline | 5.4-18.4 |
| Ellagic acid | 21.9-439.8 |
| Miquelianin | 0.2-21.4 |
| p-coumaric acid | 0.2-9.2 |
| Pipecolic acid | 1.0-74.4 |
| Tyramine | 17-182.9 |

A Rubus idaeus extract may be obtained or obtainable from raspberry fruit. The extract may be obtained by extraction with water, concentrated and spray dried.

A Fragaria extract, such as a Fragaria vesca, extract may comprise one or more of, such as all of, the following compounds given in Table 2, such as at an amount in the range given. The extract here may be a dry extract.

TABLE 2
Components of Fragaria vesca solid plant extract

| Compound | Concentration Range (μg/g = ppm) |
|---|---|
| Adenine | 206 |
| Betaine | 238 |
| Choline | 261 |
| Ellagic acid | 346 |
| p-Coumaric acid | 140 |
| Pipecolic acid | 244 |
| Miquelianin | 306 |

A Fragaria vesca extract may be obtained or obtainable from strawberry fruit. The extract may be obtained by extraction with water, concentrated and spray dried.

A Hedera extract, such as a Hedera helix extract, may be characterised by the presence of triterpene saponins, for example at an amount in the range 2.5 to 6 wt %. The extract may contain one or more of, such as all of, hederacoside C (such as in an amount in the range 1.7 to 4.8 wt %), hederacoside D (such as in an amount in the range 0.4 to 0.8 wt %), and hederacoside B (such as in an amount in the range 0.1-0.2 wt %). Additionally, or alternatively, the extract may contain α-hederin, for example at an amount in the range 0.1 to 0.3 wt %. Additionally, or alternatively, the extract may contain hederagenin, for example at an amount in the range 0.1 to 1.5 wt %, such as 0.89 or 0.9 wt %. The presence of such characterising compounds for an Hedera helix extract is as described by Tatia et al. (Rev. Chim. 2019, 70, 1157).

In one embodiment, the Hedera helix extract may be characterised by have a minimum content of hederacoside C of 3.0 wt %.

A Hedera helix extract may be obtained or obtainable from English ivy whole plant or leave.

The extract may be obtained by extraction with water, concentrated and dried.

An Ilex extract, such as an Ilex aquifolium extract, may be characterised by the presence of ursolic acid and oleanolic acid. Ursolic acid may be present at an amount of 1.30 wt %. Oleanolic acid may be present at an amount of 0.50 wt %. Additionally or alternatively, an Ilex extract may comprise amino-acids, saccharides, carotenoids, phenol derivatives, fatty acids, flavonoids, anthocyanes, and triterpenes, such as α-amyrin, β-amyrin, uvaol, and erythrodiol. The presence of such characterising compounds for an Ilex aquifolium extract is as described by Palu et al. (Molecules 2019, 24, 4413).

Ilex aquifolium extract may be obtained or obtainable from English holly whole plant or leave, such as leaf. The extract may be obtained by extraction with water, concentrated and dried.

A Garcinia extract, such as a Garcinia mangostana extract, may be characterised by the presence of one or more of, such as all of, α-mangostin, γ-mangostin and gartanin. α-Mangostin may be present at an amount in the range 8.5 to 13.9 wt %. γ-Mangostin may be present at an amount in the range 6.0 to 8.3 wt %. Gartanin may be present at an amount in the range 8.1 to 17.3 wt %. The presence of such characterising compounds for the Garcinia mangostana extract is as described by Muchtaridi et al. (J. Appl. Pharm. Sci. 2017, 7, 125).

In some embodiments, the composition of the invention comprises the plant extract in an amount of from 0.005 wt % to 50 wt %, such as based on the total weight of the composition. In a preferred embodiment, the composition of the invention comprises the plant extract in an amount of from 0.005 wt % to 30 wt %, such as 0.05 wt % to 10 wt %, more preferably 0.1 wt % to 3 wt %.

The plant extract may be present in an amount that is at most 2 wt %, 3 wt %, 5 wt %., 10 wt %, 15 wt %, 20 wt %, 25 wt %, 30 wt %, 35 wt %, 45 wt %, or 50 wt %.

The plant extract may be present in an amount that is at least 0.005 wt %, 0.01 wt %, 0.05 wt %, 0.1 wt %, 0.5 wt %, or 1 wt %.

In one embodiment, a triterpenoid compound may be substantially absent from, such as may not be present within, or otherwise derived from, the plant extract with which it is used in combination. For example, where the plant extract is a Rubus idaeus, Hehera helix or Ilex aquifolium extract, these extracts are substantially free of the triterpenoids compound, any or all triterpenoid compounds of formula (I). For example, the total triterpenoid amount may be less than 1 wt %, such as less than 0.1 wt %, such as less than 0.01 wt % in the plant extract. For example, the triterpenoid compound of formula (I) with which the plant extract is combined may be present at less than 1 wt %, such as less than 0.1 wt %, such as less than 0.01 wt % in the plant extract.

### Compositions

The compositions of the invention may be formulated for cosmetic or therapeutic uses. Accordingly, the compositions of the invention may additionally comprise one or more pharmaceutically or cosmetically ingredients.

Pharmaceutically cosmetically acceptable ingredients are those which, within the scope of sound judgment, are suitable for use in contact with the tissues of the subject in question (e.g., human) without excessive toxicity, irritation, allergic response, or other problem or complication, commensurate with a reasonable benefit/risk ratio. Each ingredient must also be compatible with the other ingredients of the composition.

In one embodiment, the composition comprises one or more ingredients selected from solvents, oils, surfactants, thickeners, humectants, and preservatives.

Examples of suitable solvents includes water; mono-alcohols such as ethanol, isopropanol, benzyl alcohol, and phenylethyl alcohol; polyalcohols such as ethylene glycol, propylene glycol, butane-1,3-diol, mannitol, sorbitol, glycerol, and erythritol; and glycol ethers such as ethylene glycol monomethyl ether and diethylene glycol monomethyl ether.

Examples of suitable oils include mineral oils, plant oils and waxes.

Examples of suitable plant oils include algal oil, annatto oil, argan oil, almond oil, apricot kernel oil, avocado oil, babassu oil, Brazil nut butter, butter, cashew butter, castor oil, camellia oil, cheery kernel oil, cocoa butter, coconut oil, corn oil, cottonseed oil, fish oil, grape seed oil, gardenia oil, ghee, hazelnut oil, jatropha oil, jojoba oil, kokum oil, linseed oil, macadamia oil, maize oil, mango seed oil, mango butter, mineral oil, mink oil, olive oil, palm oil, palm kernel oil, peach kernel 5 oil, peanut butter, peanut oil, plum kernel oil, pomegranate oil, rapeseed seed oil, rice bran oil, rosehip oil, sal oil, sesame oil, shea butter, soybean oil, squalene, sunflower oil, teas seed oil, walnut oil.

Examples of suitable waxes include bayberry wax, beeswax, carnauba wax (palm wax), candelilla wax, ceresin, jojoba butter, lanolin wax, montan wax, ozokerite, polyglyceryl-3-beeswax, polyglyceryl-6-pentastearate, Japan wax, microcrystalline wax, paraffin wax, isoparaffin, vaseline solid paraffin, squalene, oligomer olefins, synthetic candelilla wax, synthetic carnauba, synthetic beeswax

Surfactants (surface-active agents) may act as dispersants or wetting agents. Examples of suitable surfactants include anionic surfactants, cationic surfactants, non-ionic surfactants, and amphoteric (zwitterionic) surfactants.

Examples of suitable anionic surfactants include ammonium lauryl sulfate, ammonium laureth sulfate, triethylamine lauryl sulfate, triethylamine laureth sulfate, triethanolamine lauryl sulfate, triethanolamine laureth sulfate, monoethanolamine lauryl sulfate, monoethanolamine laureth sulfate, diethanolamine lauryl sulfate, diethanolamine laureth sulfate, lauric monoglyceride sodium sulfate, sodium lauryl sulfate, sodium laureth sulfate, potassium lauryl sulfate, potassium laureth sulfate, sodium lauryl sarcosinate, sodium lauroyl sarcosinate, lauryl sarcosine, cocoyl sarcosine, ammonium cocoyl sulfate, ammonium lauroyl sulfate, sodium cocoyl sulfate, sodium lauroyl sulfate, potassium cocoyl sulfate, potassium lauryl sulfate, triethanolamine lauryl sulfate, triethanolamine lauryl sulfate, monoethanolamine cocoyl sulfate, monoethanolamine lauryl sulfate, sodium tridecyl benzene sulfonate, and sodium dodecyl benzene sulfonate

Examples of suitable cationic surfactants include behentrimonium chloride, cocotrimonium chloride, cethethyldimonium bromide, dibehenyidimonium chloride, dihydrogenated tallow benzylmonium chloride, disoyadimonium chloride, ditallowdimonium chloride, hydroxycetyl hydroxyethyl dimonium chloride, hydroxyethyl behenamidopropyl dimonium chloride, Hydroxyethyl cetyidimonium chloride, hydroxyethyl tallowdimonium chloride, myristalkonium chloride, PEG-2 oleamonium chloride, PEG-5 stearmonium chloride, PEG-15 cocoyl quaternium 4, PEG-2 stearalkonium 4, lauryltrimonium chloride; Quaternium-16; Quaternium-18, lauralkonium chloride, olealkmonium chloride, cetylpyridinium chloride, Polyquaternium-5, Polyquaternium-6, Polyquaternium-7, Polyquaternium-10, Polyquaternium-22, Polyquaternium-37, Polyquaternium-39, Polyquaternium-47, cetyl trimonium chloride, dilauryidimonium chloride, cetalkonium chloride, dicetyidimonium chloride, soyatrimonium chloride, and stearyl octyl dimonium methosulfate.

Examples of suitable non-ionic surfactants include fatty alcohol ethoxylates such as octaethylene glycol monododecyl ether, and pentaethylene glycol monododecyl ether; alkylphenol ethoxylates (APEs or APEOs) such as Nonoxynol-4, Nonoxynol-7, Nonoxynol-9, Nonoxynol-14, Nonoxynol-15, Nonoxynol-18, and triton X-100; glycerol fatty acid esters such as glycerol monostearate, and glycerol monolaurate; sorbitol fatty acid esters such as sorbitan monolaurate, sorbitan monostearate, sorbitan tristearate, polysorbate (Tween) 20, polysorbate 40, polysorbate 60, and polysorbate 80.

Examples of suitable amphoteric (zwitterionic) surfactants include cocamidopropyl hydroxysultaine, cocamidopropyl betaine, lauryl betaine, lauryldimethylamine oxide, and myristamine oxide.

Examples of suitable thickeners (rheological modifiers) include gums such as alginates, carageenans, gum acacia, gum arabic, gum ghatti, gum karaya, gum tragacanth, guar gum; guar hydroxypropyltrimonium chloride, xanthan gum or gellan gum; cellulose derivatives such as sodium carboxymethyl cellulose, hydroxyethyl cellulose, hydroxymethyl carboxyethyl cellulose, hydroxymethyl carboxypropyl cellulose, ethyl cellulose, sulfated cellulose, hydroxypropyl cellulose, methyl cellulose, hydroxypropylmethyl cellulose, microcrystalline cellulose; agar; pectin; gelatin; starch and its derivatives; chitosan and its derivatives such as hydroxyethyl chitosan; synthetic polymers such as polyvinyl alcohol, PVM/MA copolymer, PVM/MA decadiene crosspolymer, poly(ethylene oxide) based thickeners:

Humectants may act as hygroscopic agents, increasing the amount of water absorbed or retained by the composition. Examples of suitable humectants include acetamide MEA, ammonium lactate, chitosan and its derivatives, colloidal oatmeal, galactoarabinan, glucose glutamate, glerecyth-7, glygeryth-12, glycereth-26, glyceryth-31, glycerin, lactamide MEA, lactamide DEA, lactic acid, methyl gluceth-10, methyl gluceth-20, panthenol, propylene glycol, sorbitol, polyethylene glycol, 1,3-butanediol, 1,2,6-hexanetriol, hydrogenated starch hydrolysate, inositol, mannitol, PEG-5 pentaerythritol ether, polyglyceryl sorbitol, xylitol, sucrose, sodium hyaluronate, and sodium PCA.

Examples of suitable preservatives includes methyl p-hydroxybenzoate, propyl p-hydroxybenzoate and sorbic acid. Methyldibromo glutaronitrile (MDBGN), benzyl alcohol, imidazolidinyl urea 1,3-bis(hydroxymethyl)-5,5-dimethyl-2,3-imidazolidinedione (DMDM hydantoin), methylchloroisothiazolinone and methylisothiazolinone, phenoxyethanol, and sodium benzoate.

In one embodiment, the composition comprises one or more ingredients selected from emollients, anti-inflammatory agents, antioxidants and UV blocking agents.

Examples of suitable emollients include fatty esters such as isopropyl myristate, isopropyl palmitate, caprylic/capric triglycerides, cetyl lactate, cetyl palmitate, hydrogenated castor oil, glyceryl esters, hydroxycetyl isostearate, hydroxy cetyl phosphate, isopropyl isostearate, isostearyl isostearate, diisopropyl sebacate, PPG-5-Ceteth-20, 2-ethylhexyl isononoate, 2-ethylhexyl stearate, C12 to C16 fatty alcohol lactate, isopropyl lanolate, and 2-ethyl-hexyl salicylate.

Examples of suitable anti-inflammatory ingredients include cyclo-oxygenase (e.g., COX-1 or COX-2) or Lipoxygenase (e.g., LOX-5) enzyme inhibitors such as ascorbic acid, ascorbic acid derivatives, vitamin E, vitamin E derivatives, tocotrienol, rutin, quercetin, hesperidin (Citrus sinensis), hesperetin (Citrus sinensis), diosmin (Citrus sinensis), mangiferin (Mangifera indica), mangostin (Garcinia mangostana), cyanidin (Vaccinium myrtillus), astaxanthin (Haematococcus algae), lutein (Tagetes patula), lycopene (Lycopersicum esculentum), resveratrol (Polygonum cuspidatum), tetrahydrocurcumin (Curcuma longa), rosmarinic acid (Rosmarinus officinalis), hypericin (Hypericum perforatum), ellagic acid (Punica granatum), chlorogenic acid (Vaccinium vulgaris), oleuropein (Olea europaea), alpha-lipoic acid, glutathione, andrographolide (Andrographis paniculata), grapeseed extract, green tea extract, polyphenols, pycnogenol (pine bark extract), white tea extract, black tea extract, (Andrographis paniculata), carnosine, and niacinamide. Further examples of suitable anti-inflammatory composition can additionally be selected from horse chestnut extract (Aesculus hippocastanum extract), esculin, escin, yohimbine, Capsicum oleoresin, capsaicin, niacin, niacin esters, methyl nicotinate, benzyl nicotinate, ruscogenins (Butchers Broom extract; Ruscus aculeatus extract), diosgenin (Trigonel afoenumgraecum, fenugreek), emblica extract (Phyilanthus emblica extract), asiaticoside (Centella asiatica extract), Boswellia extract (Boswellia serrata), sericoside, visnadine, thiocolchicoside, grapeseed extract, ginger root extract (Zingiber officianale), piperine, vitamin K, melilot (Melilotus officinalis extract), glycyrrhetinic acid, ursolic acid, sericoside (Terminalia sericea extract), darutoside (Siegesbeckia orientalis extract), Amni visnaga extract, vine leaf extract (Vitis vinifera), apigenin, phytosan, luteolin.

Examples of suitable antioxidant ingredients include ascorbic acid, ascorbic acid derivatives glucosamine ascorbate, arginine ascorbate, lysine ascorbate, glutathione ascorbate, nicotinamide ascorbate, niacin ascorbate, allantoin ascorbate, creatine ascorbate, creatinine ascorbate, chondroitin ascorbate, chitosan ascorbate, carnosine ascorbate, vitamin E, vitamin E derivatives, tocotrienol, rutin, quercetin, hesperidin (Citrus sinensis), hesperetin (Citrus sinensis), diosmin (Citrus sinensis), mangiferin (Mangifera indica), mangostin (Garcinia mangostana), cyanidin (Vaccinium myrtillus), astaxanthin (Haematococcus algae), lutein (Tagetes patula), lycopene (Lycopersicum esculentum), resveratrol (Polygonum cuspidatum), tetrahydrocurcumin (Curcuma longa), rosmarinic acid (Rosmarinus officinalis), hypericin (Hypericum perforatum), ellagic acid (Punica granatum), chlorogenic acid (Vaccinium vulgaris), oleuropein (Olea europaea), alpha-lipoic acid, niacinamide lipoate, glutathione, andrographolide (Andrographis paniculata), carnosine, niacinamide, Potentilla erecta extract, polyphenols, grapeseed extract, pycnogenol (pine bark extract), pyridoxine, magnolol, honokiol, paeonol, resacetophenone, quinacetophenone, arbutin and kojic acid.

Examples of suitable UV blocking agents (sunscreen active agents) include octyl methoxycinnamate (ethylhexyl p-methoxycinnamate), octyl salicylate oxybenzone (benzophenone-3), benzophenone-4, menthyl anthranilate, dioxybenzone, aminobenzoic acid, amyl dimethyl PABA, diethanolamine p-methoxy cinnamate, ethyl 4-bis(hydroxypropyl) aminobenzoate, 2-ethylhexyl-2-cyano-3,3-diphenylacrylate, homomenthyl salicylate, glyceryl aminobenzoate, dihydroxyacetone, octyl dimethyl PABA, 2-phenylbenzimidazole-5-sulfonic acid, triethanolamine salicylate, zinc oxide, titanium oxide, and mixtures thereof.

Further components suitable for use in the composition include fragrances, pH adjusters, pigments, odour absorbers, antimicrobial agents, antifungal agents, chelating agents, and saccharides.

The composition of the present invention are typically formulated for topical use. The composition of the present invention may be formulated as a solution, liquid, lotion, cream, emulsion, dispersion, gel, or paste. Examples of suitable emulsions include two-phase emulsions comprising an aqueous phase and an oil phase such as oil-in-water (O/W) and water-in-oil (W/O), as well as complex emulsions such as triple emulsions (O/W/O and W/O/W).

The composition and formulations may be prepared by any methods well known in the art. Such methods include the step of bringing into association the triterpenoid compound and/or plant extract with a carrier which constitutes one or more accessory ingredients. In general, the formulations are prepared by uniformly mixing the triterpenoid compound and/or plant extract with a carrier (e.g., a liquid carrier, a finely divided solid carrier, etc.).

The compositions of the present invention can be formulated as both cosmetic and pharmaceutical products.

In one embodiment, there is provided a personal care product comprising a composition of the invention. Suitable personal care products include skin care products, hair care products, cleansing products, and cosmetic powders and liquids.

Examples of suitable skin care products include skin/hand lotions, skin/hand creams, skin/hand ointments, skin/hand pastes, skin toner, shaving gels, shaving creams, sunscreens, deodorants, antiperspirants, suntan lotions, after sun, aftershaves, body oils, bath oils, and bubble baths. Examples of suitable hair care products include conditioners, hair detangling lotion, styling gel, styling creams, styling waxes, styling lotions, mousses, spray gels, hair tonics, spritzes, and pomades. Examples of suitable cleansing products include liquid soaps, bar soaps, body washes, skin cleansers, and shampoos. Examples of suitable cosmetic powders and liquids include blusher, body powder, bronzing powder, eye shadow, foundation, face powder, lip powder, powder makeup, liquid bronzer, eyeliner, lip gloss, lipstick, and mascara.

### Method and Uses for Decreasing Lipid Production

The compositions of the invention reduce the production of lipids in sebocytes.

The invention also provides a method for decreasing lipid production in sebocytes, the method comprising contacting the sebocytes with a composition of the invention, such as a composition comprising:
(a) a triterpenoid compound according to formula (I), or a pharmaceutically or cosmetically acceptable salt, prodrug, solvate, tautomer or stereoisomer thereof; and
(b) a plant extract selected from extracts of plants belonging to the genus Garcinia, Rubus, Fragaria, lex and Hedera.

Preferred features of the composition, such as the triterpenoid compound and the plant extract, are set out above.

In one embodiment, the method for decreasing lipid production in sebocytes is in vivo. In one embodiment, the method for decreasing lipid production in sebocytes is ex vivo, such as in vitro.

In one embodiment, the method for decreasing lipid production in sebocytes comprises contacting the sebocytes with an effective amount of a composition of the invention. An effective amount of the composition provides a detectable reduction in the production of lipids in the sebocytes. Methods for detecting and quantifying the production of lipids in sebocytes include, for example, a fluorescence assay using a suitable lipid-detecting dye, such as AdipoRed™.

In one embodiment, the method for decreasing lipid production in sebocytes comprises contacting the sebocytes with an amount of a composition of the invention suitable for reducing the production of lipids by 10% or more, preferably 15% or more, more preferably 20% or more, even more preferably 20% or more, and most preferably 30% or more.

In one embodiment, the method for decreasing lipid production in sebocytes decreases lipid production by 5% or more, preferably 10% or more, preferably 15% or more, more preferably 20% or more, even more preferably 20% or more, and most preferably 30% or more.

The invention also provides a composition of the invention, such as a composition comprising:
(a) a triterpenoid compound according to formula (I), or a pharmaceutically or cosmetically acceptable salt, prodrug, solvate, tautomer or stereoisomer thereof; and
(b) a plant extract selected from extracts of plants belonging to the genus Garcinia, Rubus, Fragaria, Ilex and Hedera,
   for use in a method of decreasing lipid production in sebocytes.

Preferred features of the composition, such as the triterpenoid compound and the plant extract, are set out above. Preferred features of the method for decreasing lipid production in sebocytes are also set out above.

The invention also provides the use of a composition of the invention, such as a composition comprising:
(a) a triterpenoid compound according to formula (I), or a pharmaceutically or cosmetically acceptable salt, prodrug, solvate, tautomer or stereoisomer thereof; and
(b) a plant extract selected from extracts of plants belonging to the genus Garcinia, Rubus, Fragaria, Ilex and Hedera,
   to decrease lipid production in sebocytes.

Preferred features of the composition, such as the triterpenoid compound and the plant extract, are set out above. Preferred features of the method for decreasing lipid production in sebocytes also apply to the use of the composition to decrease lipid production in sebocytes.

### Cosmetic Methods and Uses

The compositions of the invention reduce lipid production in sebocytes, such as those in the skin of an individual. As such, the compositions of the invention can reduce or ameliorate cosmetic problems associated with over-production of lipids in the skin. Cosmetic problems associated with overproduction of lipids in the skin include oily or shiny skin, oily hair, enlarged skin pores, undesirable body odour, and decreased retention of make-up products on the skin.

Accordingly, the invention provides a cosmetic method for decreasing lipid production in the skin of an individual, the method comprising contacting the skin with a composition of the invention, such as a composition comprising:
(a) a triterpenoid compound according to formula (I), or cosmetically acceptable salt, prodrug, solvate, tautomer or stereoisomer thereof; and
(b) a plant extract selected from extracts of plants belonging to the genus Garcinia, Rubus, Fragaria, Ilex and Hedera.

Preferred features of the composition, such as the triterpenoid compound and the plant extract, are set out above.

In one embodiment, the cosmetic method for decreasing lipid production in the skin of an individual is not a method of treatment. In one embodiment, the cosmetic method for decreasing lipid production in the skin of an individual is not a method of treatment of the human or animal body by therapy.

In one embodiment, the cosmetic method reduces lipid production in sebocytes in the skin of an individual.

In one embodiment, the skin is skin on the head, such as on the face, mouth, neck, or scalp. In one embodiment, the skin is skin on the chest, back, arms, legs, or hands.

The benefits of decreasing lipid production in the skin of an individual include reducing the oily appearance of the skin, controlling surface oil, minimizing skin pores and reducing undesirable body odour. In one embodiment, the cosmetic method decreases lipid production in the skin, thereby achieving an effect selected from reducing the oily appearance of the skin, controlling skin surface oil, minimizing skin pores and reducing undesirable body odour.

In one embodiment, the individual is in need of cosmetic treatment. Individuals in need of cosmetic treatment may have a condition associated with overproduction of lipids in the skin, such as oily or shiny skin, oily hair, enlarged skin pores, and undesirable body odour. In one embodiment, the individual has a cosmetic condition selected from oily or shiny skin, oily hair, enlarged skin pores, and undesirable body odour.

The invention also provides a composition of the invention, such as a composition comprising:
(a) a triterpenoid compound according to formula (I), or a pharmaceutically or cosmetically acceptable salt, prodrug, solvate, tautomer or stereoisomer thereof; and
(b) a plant extract selected from extracts of plants belonging to the genus Garcinia, Rubus, Fragaria, Ilex and Hedera, for use in a cosmetic method of decreasing lipid production in the skin of an individual.

Preferred features of the composition, such as the triterpenoid compound and the plant extract, are set out above. Preferred features of the cosmetic method for decreasing lipid production in the skin of an individual are also set out above.

The invention also provides the use of a composition of the invention, such as a composition comprising:
(a) a triterpenoid compound according to formula (I), or a pharmaceutically or cosmetically acceptable salt, prodrug, solvate, tautomer or stereoisomer thereof; and
(b) a plant extract selected from extracts of plants belonging to the genus Garcinia, Rubus, Fragaria, Ilex and Hedera,
   to decrease lipid production in the skin of an individual.

Preferred features of the composition, such as the triterpenoid compound and the plant extract, are set out above. Preferred features of the cosmetic method for decreasing lipid production in the skin of an individual also apply to the use of the composition to decrease lipid production in the skin of an individual.

### Medical Methods and Uses

The compositions of the invention reduce lipid production in sebocytes, such as those in the skin of an individual. As such, the compositions of the invention are useful in the treatment or prophylaxis (prevention) of medical problems associated with over-production of lipids in the skin. Medical problems associated with overproduction of lipids in the skin include acne vulgaris and rosacea.

Accordingly, the invention provides a composition of the invention, such as a composition comprising:
(a) a triterpenoid compound according to formula (I), or a pharmaceutically or cosmetically acceptable salt, prodrug, solvate, tautomer or stereoisomer thereof; and
(b) a plant extract selected from extracts of plants belonging to the genus Garcinia, Rubus, Fragaria, Ilex and Hedera,
   for use in a method of treatment, such as a method of treatment of the human or animal body by therapy.

In one embodiment, the method of treatment is a method of treatment of a disorder (e.g. a disease) associated with over-production of lipids in the skin. In one embodiment, the method of treatment is a method of treatment of a disorder associated with over-production of lipids in sebocytes.

In one embodiment, the skin is skin on the head, such as on the face, mouth, neck, or scalp. In one embodiment, the skin is skin on the chest, back, arms, legs, or hands.

In one embodiment, the treatment is treatment of acne vulgaris. In one embodiment, the treatment is treatment of rosacea.

In one embodiment, the treatment is administered to a subject in need of treatment.

The subject in need of treatment (the patient) may be a mammal, such as a human.

The subject in need of treatment may be an adult or juvenile.

In a preferred embodiment, the subject in need of treatment is a human, more preferably an adult human.

Alternatively, the subject in need of treatment is a non-human animal used in laboratory research.

In one embodiment, the treatment is administered by any convenient route of administration.

In a preferred embodiment, the treatment is administered topically (i.e. at the site of desired action).

In one embodiment, the treatment comprises administering a therapeutically-effective amount of the composition to a subject in need of treatment.

It will be appreciated by one of skill in the art that appropriate dosages of the compositions described herein, can vary from patient to patient. Determining the optimal dosage will generally involve the balancing of the level of therapeutic benefit against any risk or deleterious side effects. The selected dosage level will depend on a variety of factors including, but not limited to, the activity of the particular triterpenoid compound and plant extract, the route of administration, the time of administration, the rate of excretion of the triterpenoid compound and plant extract, the duration of the treatment, other drugs, compounds, and/or materials used in combination, the severity of the disorder, and the species, sex, age, weight, condition, general health, and prior medical history of the patient. The amount of triterpenoid compound and plant extract and route of administration will ultimately be at the discretion of the physician, veterinarian, or clinician, although generally the dosage will be selected to achieve local concentrations at the site of action which achieve the desired effect without causing substantial harmful or deleterious side-effects.

Administration can be effected in one dose (application), continuously or intermittently (e.g., in divided doses at appropriate intervals) throughout the course of treatment. Methods of determining the most effective means and dosage of administration are well known to those of skill in the art and will vary with the formulation used for therapy, the purpose of the therapy, the target cell(s) being treated, and the subject being treated. Single or multiple administrations can be carried out with the dose level and pattern being selected by the treating physician, veterinarian, or clinician.

### Other Preferences

Each and every compatible combination of the embodiments described above is explicitly disclosed herein, as if each and every combination was individually and explicitly recited.

Various further aspects and embodiments of the invention will be apparent to those skilled in the art in view of the present disclosure.
“and/or” where used herein is to be taken as specific disclosure of each of the two specified features or components with or without the other. For example, “A and/or B” is to be taken as specific disclosure of each of (i) A, (ii) B and (iii) A and B, just as if each is set out individually herein.

Unless context dictates otherwise, the descriptions and definitions of the features set out above are not limited to any particular aspect or embodiment of the invention and apply equally to all aspects and embodiments which are described.

### EXAMPLES

Certain aspects and embodiments of the invention will now be illustrated by way of example and with reference to the figures described above.

### Materials

Dimethyl sulfoxide (DMSO), epigallocatechin gallate (EGCG), and salicylic acid (SA) were purchased from Sigma-Aldrich. Oleanolic acid (OA), Beta-elemonic acid and Cycloastragenol were purchased from TargetMol (Boston, USA).

Rubus idaeus extract (RI), Hedera helix extract (HH), Ilex aquifolium extract (IA), Fragaria vesca extract (FV) and Garcinia mangostana extract (GM) were purchased from BOCSCl (New York, USA).

Primary sebocytes were derived from the forehead of a 48-year-old Chinese male. The same cell line was used in all experiments.

### Characterisation Methods

Flow cytometry experiments were conducted on the BD LSR Fortessa X-20 as follows: Sebocytes stained with a lipid dye (AdipoRed™) were first analyzed according to size and granularity with only single cells selected for AdipoRed™ signal intensity measurement. The AdipoRed™ signal was measured using the 488 nm FITC blue laser and the value for the average fluorescence intensity of 10,000 cells obtained. The average fluorescence intensity is a measure of the average lipid quantity in 10,000 sebocytes. Cells treated with compositions of the invention were compared to vehicle-treated cells to determine the percentage of lipid reduction.

### Primary Sebocytes Culture

Primary sebocytes were cultured to 80% confluency in Complete Culture Medium with Y-27632 (CCMY) consisting of 3:1 DMEM (Gibco, 11995-065)/F12 (Gibco, 31765-035), supplemented with 10% fetal bovine serum (Hyclone, SV30160.03), 1× Penstrep (Gibco, 15140-122), 0.2 μg/mL Epidermal Growth Factor (PeproTech, AF-100-15-1MG), 1 μg/mL Hydrocortisone (Sigma-Aldrich, H0888), 10⁻⁹ M cholera toxin (Sigma-Aldrich, C8052-2MG) and 10 μM Y-27632 (Tocris, 1254/10) at 37° C. with 5% CO₂. The cells were washed with PBS buffer [5 mL for a 10 cm culture plate] and then incubated at 37° C. with 0.125% Trypsin-EDTA (Gibco, 15400054) [2 mL of Trypsin for a 10 cm culture plate] for 5-10 minutes. When all the cells had detached, neutralisation media consisting of DMEM with 10% FBS and 1× Penstrep (2 mL of neutralisation media for a 10 cm culture plate) was added, and the cells were transferred to a 15 mL conical tube and centrifuged at 1,000 rpm for 5 minutes.

After centrifugation, a cell pellet was formed, the supernatant was discarded and the cells were resuspended in culture medium.

The cells were cultured in a CelCulture CO₂ Incubator 170L (Esco Lifesciences).

Cells were counted by loading onto a hemocytometer, with the cells observed using a CKX41 Inverted Microscope (Olympus) and manually counted.

### General Test Protocol

A test composition was added to the well of a 24 well plate to achieve a final concentration of 10 μM for the compound (4 μL of a 10 mM stock solution made up in DMSO into 396 μL of culture media) or 31.25 μg/mL for the extract (4 μL of a 3,125 μg/mL stock solution made up in DMSO into 396 μL of culture media).

Primary sebocytes were seeded at 60×10⁶ cells per well (396 μL) in culture media. The cells were incubated for 3 days at 37° C. and 5% CO₂. The media was discarded, AdipoRed™ dye (0.06% v/v in PBS) was added and the cells incubated at 37° C. for 20 minutes. The staining solution was discarded, trypsin (0.125%; 250 μL) was added and the cells incubated at 37° C. for 5-10 minutes. When all cells had detached, fixative solution (250 μL; MEM without phenol red, 10% fetal bovine serum and 4% paraformaldehyde) was added and the cells transferred to 5 mL polystyrene tubes. The tubes were stored on ice before flow cytometry. Mean fluorescence intensity of the lipid signal can be obtained from the flow cytometer plot which are used to determine the inhibitory effect of the compounds on the sebocytes.

### Example 1

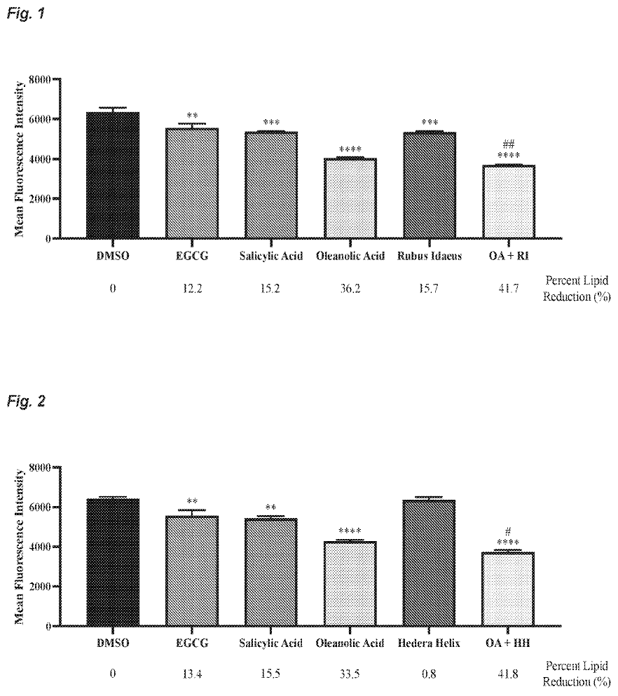

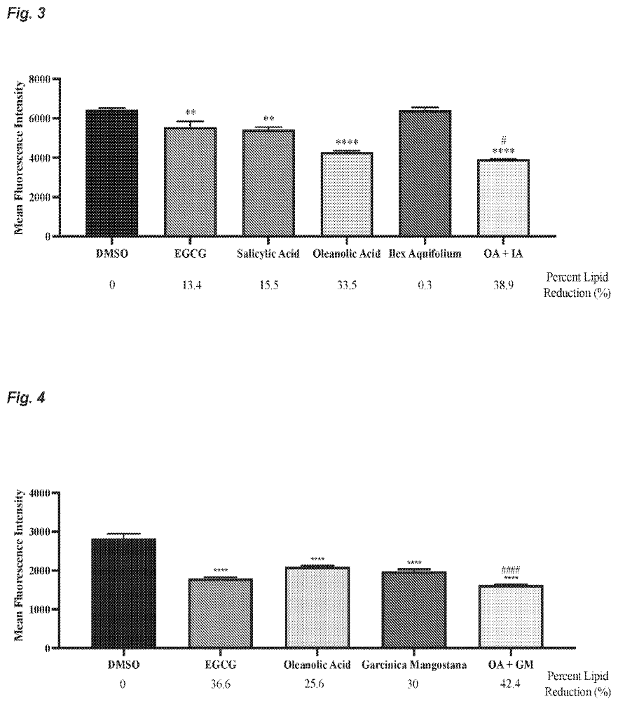

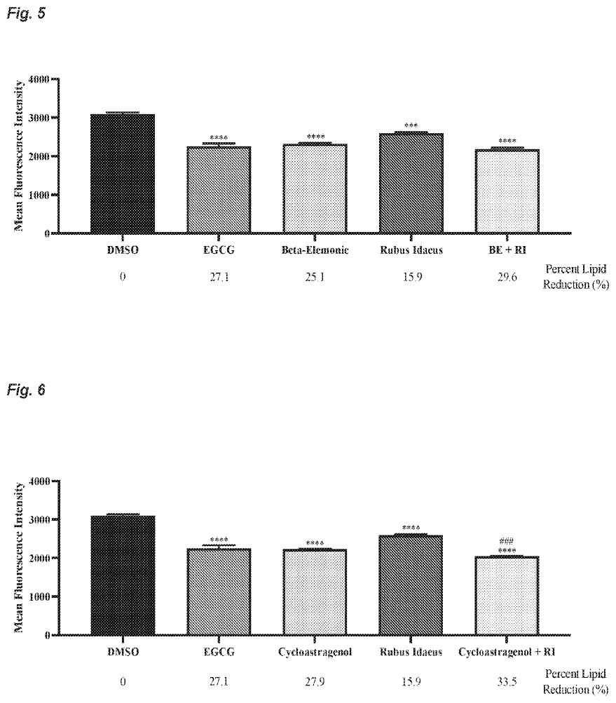

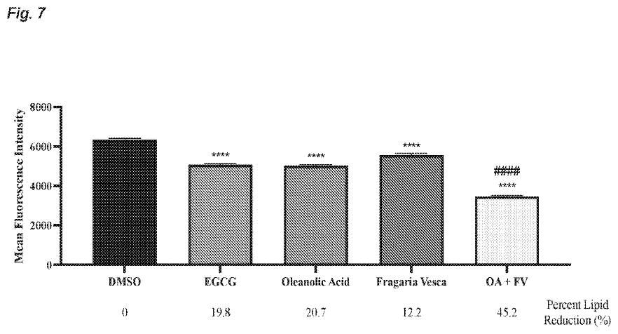

The lipid reduction effects of the compositions of the invention were measured against DMSO as a negative control, and against the known lipid-reducing compounds epigallocatechin gallate (EGCG) and salicylic acid (SA) as positive controls. The results are shown in Table 3 and FIGS. 1 to 7.

The results demonstrate that a composition comprising a triterpenoid compound (oleanoic acid (OA)) and a plant extract (Rubus idaeus extract (RI), Hedera helix extract (HI), Ilex aquifolium extract (IA), Fragaria vesca extract (FV) and Garcinia mangostana extract (GM)) provides improved lipid reduction in comparison to the individual compound or extract. The results demonstrate a synergist effect for the composition against the individual compounds.

TABLE 3
Percentage reduction in mean fluorescence intensity vs DMSO

| | | Percentage Reduction in |
|---|---|---|
| | | Mean Fluorescence Intensity |
| Entry | Composition | (Against DMSO Control) |
| 1 | DMSO | 00.0 |
| 2 | EGCG | 13.4 |
| 3 | SA | 15.5 |
| 4 | OA | 33.5 |
| 5 | RI | 15.7 |
| 6 | HH | 0.8 |
| 7 | IA | 0.3 |
| 8 | GM | 30.0 |
| 9 | FV | 12.2 |
| 10 | OA + RI | 41.7 |
| 11 | OA + HH | 41.8 |
| 12 | OA + IA | 38.9 |
| 13 | OA + GM | 42.4 |
| 14 | OA + FV | 45.2 |

### REFERENCES

A number of publications are cited above in order to more fully describe and disclose the invention and the state of the art to which the invention pertains. Full citations for these references are provided below. The entirety of each of these references is incorporated herein.
Cheng et al. Int. J. Biochem. Cell Biol., 2010: Vol. 42, pp. 181-185
Muchtaridi et al. J. Appl. Pharm. Sci. 2017, 7, 125
Palu et al. Molecules 2019, 24, 4413
Tatia et al. Rev. Chim. 2019, 70, 1157
WO 2020/263188

1. A composition comprising:
(a) a triterpenoid compound according to formula (I), or a pharmaceutically or cosmetically acceptable salt, prodrug, solvate, tautomer or stereoisomer thereof; and
(b) a plant extract selected from extracts of plants belonging to the genus Garcinia, Rubus, Fragaria, Ilex and Hedera, 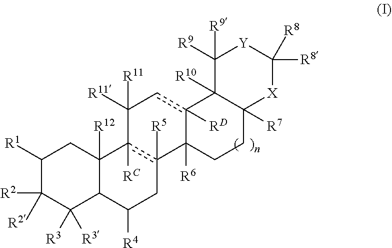
wherein:
—Y— is a covalent bond and —X— is —CH₂—, —CH₂CH₂—, or —X— is absent, and the carbon atoms to which —X— is connected are bonded to H, or
—Y— is —O—, and —X— is absent, and the carbon atoms to which —X— is connected are bonded to H,
a dashed bond indicates the presence of a double or single bond between the ring carbon atoms, where only one double bond is present within the ring,
R¹, R³, R^{3′}, R⁴, R⁵, R⁶, R⁷, R⁸, R^{8′}, R⁹, R^{9′}, R¹⁰, and R¹² are independently selected from H, F, OH, SH, C₁₋₆ alkyl, C₁₋₄haloalkyl, C₁₋₄ hydroxyalkyl C₂₋₆ alkenyl, OR¹³, CO₂R¹⁴, OC(═O)R¹⁵ and, where —Y— is —O— and —X— is absent, R⁸ and R⁹ may together form a heterocycle, and R⁵ is absent when the ring carbon to which it is connected is in a double bond;
R² and R^{2′} are independently selected from H, F, OH, SH, C₁₋₆ alkyl, C₁₋₄ haloalkyl, C₂₋₆ alkenyl, OR¹⁶, CO₂R¹⁷, OC(═O)R¹⁸, or R² and R^{2′} together represent ═O;
R¹¹ and R^{11′} are independently selected from H, F, OH, SH, C₁₋₆ alkyl, I, C₁₋₄ haloalkyl, C₂₋₆ alkenyl, OR¹⁹, CO₂R²⁰, OC(═O)R²¹, or R¹¹ and R^{11′} together represent ═O;
R^{C} is absent, or where the ring carbon to which it is connected is not in a double bond, R^{C} is H, F, OH, SH, C₁₋₆ alkyl, C₁₋₄ haloalkyl, C₁₋₄ hydroxyalkyl, C₂₋₆ alkenyl, OR¹³, CO₂R¹⁴, OC(═O)R¹⁵, or together with R¹² may form a carbocycle;
R^{D} is absent, or where the ring carbon to which it is connected is not in a double bond, R^{D} is H, F, OH, SH, C₁₋₆ alkyl, C₁₋₄ haloalkyl, C₁₋₄ hydroxyalkyl, C₂₋₆ alkenyl, OR¹³, CO₂R¹⁴, OC(═O)R¹⁵,
R¹³, R¹⁴, R¹⁵, R¹⁶, R¹⁷, R¹⁸ R¹⁹, R²⁰, and R²¹ are independently selected from H, C₁₋₆ alkyl and C₁₋₄ haloalkyl; and
n is 0 or 1.

2. The composition of claim 1, wherein:
—Y— is a covalent bond and —X— is —CH₂— or —CH₂CH₂—, and
n is 1.

3. The composition of claim 1, wherein:
—Y— is a covalent bond or —O—, and —X— is absent, and the carbon atoms to which —X— is connected are bonded to H, and
n is 0.

4. The composition of claim 1, wherein:
R¹, R³, R^{3′}, R⁴, R⁵, R⁶, R⁷, R⁸, R^{8′}, R⁹, R^{9′}, R¹⁰, and R¹² are independently selected from H, F, OH, SH, OR¹³, CO₂R¹⁴, OC(═O)R¹⁵, C₁₋₆ alkyl, CF₃, and C₂₋₆ alkenyl;
R² and R^{2′} are independently selected from H, OH, OMe, OEt, C₁₋₄ alkyl, C₂₋₄ alkenyl, CO₂H, CO₂Me, CO₂Et, OC(O)Me, and OC(O)Et, or R², R^{2′} together form ═O;
R¹¹ and R^{11′} are independently selected from H, OH, OMe, OEt, C₁₋₄ alkyl, C₂₋₄ alkenyl, CO₂H, CO₂Me, CO₂Et, OC(O)Me, and OC(O)Et, or R² and R^{2′} together form ═O;
R¹³, R¹⁴, and R¹⁵ are independently selected from H, C₁₋₆ alkyl, and CF₃; and
n is 1 or 2.

5. The composition of claim 2, wherein:
R¹, R³, R^{3′}, R⁴, R⁵, R⁶, R⁷, R⁸, R^{8′}, R⁹, R^{9′}, R¹⁰, and R¹² are independently selected from H, F, OH, OR¹³, CO₂R¹⁴, OC(═O)R¹⁵, C₁₋₆ alkyl, and C₂₋₆ alkenyl;
R² and R^{2′} are independently selected from H, OH, OMe, OEt, C₁₋₄ alkyl, C₂₋₄ alkenyl, CO₂H, CO₂Me, CO₂Et, OC(O)Me, and OC(O)Et, or R² and R^{2′} together form ═O;
R¹¹ and R^{11′} are independently selected from H, OH, OMe, OEt, C₁₋₄ alkyl, C₂₋₄ alkenyl, CO₂H, CO₂Me, CO₂Et, OC(O)Me, and OC(O)Et, or R², R^{2′} together form ═O;
R¹³, R¹⁴, and R¹⁵ are independently selected from H and C₁₋₆ alkyl; and
n is 1 or 2.

6. The composition of claim 5, wherein:
R¹, R³, R^{3′}, R⁴, R⁵, R⁶, R⁷, R⁸, R^{8′}, R⁹, R^{9′}, R¹⁰, and R¹² are independently selected from H, OH, Me, Et, vinyl, allyl, 1-methylvinyl, OMe, OEt, CH₂OH, CH₂CH₂OH, CO₂H, CO₂Me, and OC(O)Me;
R² and R^{2′} are independently selected from H, OH, Me, Et, vinyl, allyl, 1-methylvinyl, OMe, CO₂H, CO₂Me, and OC(O)Me, or R², R^{2′} together represent ═O;
R¹¹ and R^{11′} are independently selected from H, OH, Me, Et, vinyl, allyl, 1-methylvinyl, OMe, CO₂H, CO₂Me, and OC(O)Me, or R² and R^{2′} together represent ═O; and
n is 1 or 2.

7. The composition of claim 1, wherein:
R¹ and/or R⁴ and/or R¹⁰ are H.

8. The composition of claim 1, wherein:
R⁵ and/or R⁶ and/or R¹² are Me.

9. The composition of claim 6, wherein:
R⁷ is CO₂H.

10. The composition of claim 1, wherein the triterpenoid compound is a compound of formula (IIa): 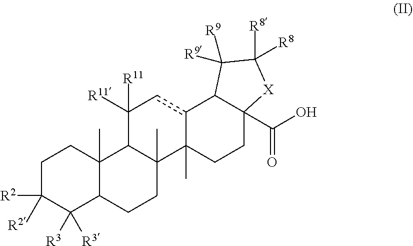
wherein:
R³, R^{3′}, R⁸, R^{8′}, R⁹, and R^{9′} are independently selected from H, OH, Me, Et, vinyl, allyl, 1-methylvinyl, OMe, OEt, CH₂OH, CH₂CH₂OH, CO₂H, CO₂Me, and OC(O)Me;
R² and R^{2′} are independently selected from H, OH, Me, Et, vinyl, allyl, 1-methylvinyl, OMe, OEt, CH₂OH, CH₂CH₂OH, CO₂H, CO₂Me, and OC(O)Me, or R² and R^{2′} together represent ═O;
R¹¹ and R^{11′} are independently selected from H, OH, Me, Et, vinyl, allyl, 1-methylvinyl, OMe, OEt, CH₂OH, CH₂CH₂OH, CO₂H, CO₂Me, and OC(O)Me, or R¹¹ and R^{11′} together represent ═O;
X is —CH₂—, —CH₂CH₂—; and
a dashed bond indicates the presence of a double or single bond between the ring carbon atoms.

11. The composition of claim 10, wherein:
R³, R^{3′}, R⁸, R^{8′} R¹¹ and R^{11′} are independently selected from H, OH, Me, OMe, CH₂OH, and CO₂H;
R² and R^{2′} are independently selected from H, OH, Me, OMe, CH₂OH, CO₂H and OC(O)Me, or R² and R^{2′} together represent ═O;
R⁹, and R^{9′} H, OH, Me, 1-methylvinyl, OMe, CH₂OH, and CO₂H.

12. The composition of claim 1, wherein the triterpenoid compound is selected from a compound of formulae (II) to (VII):

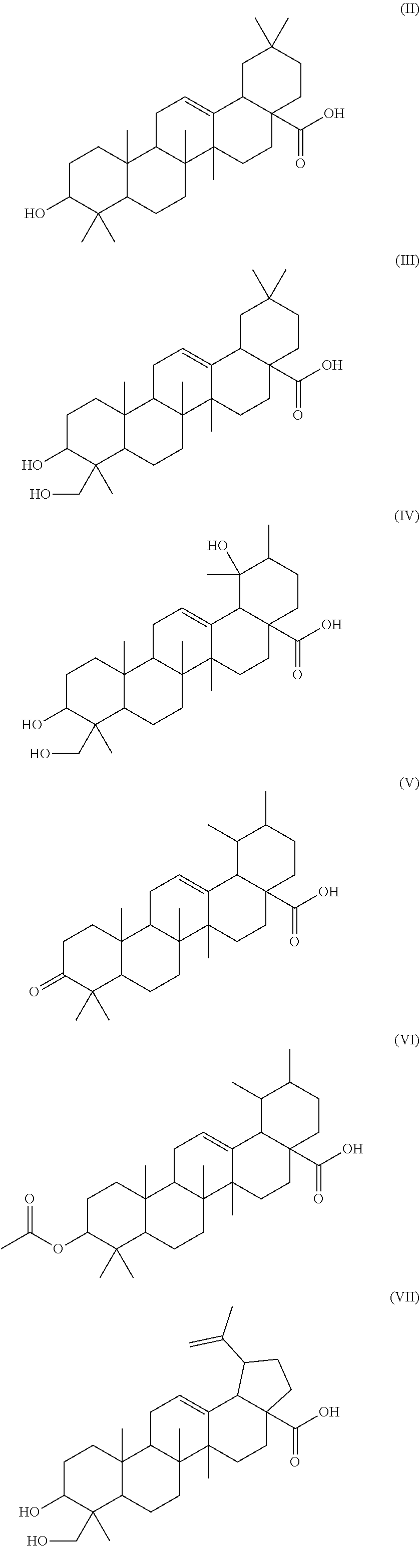

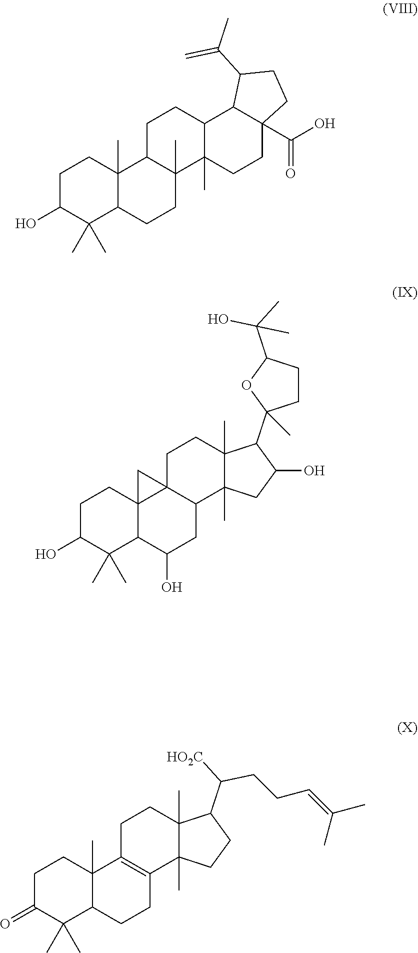

13. The composition of claim 12, wherein the triterpenoid compound is selected from, oleanolic acid, hederagenin, rotundic acid, ursonic acid, acetylursolic acid, anemosapogenin, betulinic acid (lupatic acid), cycloastragenol, and β-elemonic acid (elemadienonic acid).

14. The composition of claim 1, wherein the plant extract is selected from Rubus idaeus extract, Garcinia mangostana extract, Hedera helix extract, Ilex aquifolium leaf extract and Fragaria vesca extract.

15. The composition of claim 1, wherein the triterpenoid compound is oleanolic acid and the plant extract is selected from extracts of plants belonging to the genus Garcinia, Rubus, Fragaria, Ilex and Hederal, such as selected from Rubus Idaeus extract, Hedera helix extract, Ilex aquifolium, Garcinia mangostana and Fragaria Vesca.

16. (canceled)

17. (canceled)

18. (canceled)

19. The composition of claim 1, wherein the triterpenoid compound is present in an amount of from 0005 wt % to 10 wt %, preferably 0.1 wt % to 5 wt %, more preferably 0.1 wt % to 3 wt %, such as 0.2 wt % to 2 wt %.

20. The composition of claim 1, wherein the plant extract is present in an amount of from 0.005 wt % to 50 wt %, preferably 0.005 wt % to 30 wt %, such as 0.05 wt % to 10 wt %, and more preferably 0.1 wt % to 3 wt %.

21. (canceled)

22. (canceled)

23. (canceled)

24. (canceled)

25. (canceled)

26. A method for decreasing lipid production in sebocytes or in the skin of an individual, the method comprising contacting the skin with the composition of claim 1, wherein the method is not a method of treatment.

27. (canceled)

28. The composition of claim 1 for use in decreasing lipid production in sebocytes.

29. (canceled)

30. (canceled)

31. (canceled)

32. Use of the composition of claim 1 for decreasing lipid production in sebocytes.
